(19) United States
(12) Patent Application Publication
(10) Pub. No.: US 2011/0105536 A1
(43) Pub. Date: May 5, 2011

(54) DOSING REGIMEN ASSOCIATED WITH LONG-ACTING INJECTABLE PALIPERIDONE ESTERS

(76) Inventors: Peter H. Lewyn-Briscoe, Newtown, PA (US); Cristiana Gassmann-Mayer, Pennington, NJ (US); Srihari Gopal, Belle Meade, NJ (US); David W. Hough, Wallingford, PA (US); Bart M.M. Remmerie, Gent (BE); Mahesh N. Samtani, Flemington, NJ (US)

(21) Appl. No.: 12/916,910

(22) Filed: Nov. 1, 2010

Related U.S. Application Data
(60) Provisional application No. 61/256,696, filed on Oct. 30, 2009.

(51) Int. Cl. A61K 31/519; A61P 25/18

(52) U.S. Cl. 514/259.41

(57) ABSTRACT

The present application provides a method for treating patients in need of psychiatric treatment, wherein said patient misses a stabilized dose of a monthly maintenance regimen of paliperidone palmitate. The present application also provides a method for treating psychiatric patients in need of a switching treatment to paliperidone palmitate in a sustained release formulation.

### CROSS REFERENCE TO RELATED APPLICATIONS

This application claims the benefit of U.S. Provisional Application 61/256,696, filed on Oct. 30, 2009, which is incorporate by reference herein in its entirety.

### FIELD OF THE INVENTION

This invention relates to a method for treating patients in need of switching treatment from other antipsychotic drug to long-acting injectable paliperidone palmitate formulations.

### BACKGROUND OF THE INVENTION

Antipsychotic medications are the mainstay in the treatment of schizophrenia, schizoaffective disorder, and schizophreniform disorders. Conventional antipsychotics were introduced in the mid-1950s. These typical or first generation drugs are usually effective in controlling the positive symptoms of schizophrenia, but are less effective in moderating the negative symptoms or the cognitive impairment associated with the disease. Atypical antipsychotics or second generation drugs, typified by risperidone and olanzapine, were developed in the 1990s, and are generally characterized by effectiveness against both the positive and negative symptoms associated with schizophrenia.

Paliperidone palmitate is the palmitate ester of paliperidone (9-hydroxy-risperidone), a monoaminergic antagonist that exhibits the characteristic dopamine D₂ and serotonin (5-hydroxytryptamine type 2A) antagonism of the second generation, atypical antipsychotic drugs. Paliperidone is the major active metabolite of risperidone. Extended release (ER) osmotic controlled release oral delivery (OROS) paliperidone, as a tablet formulation, is marketed in the United States (U.S.) for the treatment of schizophrenia and maintenance of effect.

Paliperidone palmitate is being developed as a long-acting, intramuscular (i.m.), injectable aqueous nanosuspension for the treatment of schizophrenia and other related diseases that are normally treated with antipsychotic medications. Because of extreme low water solubility, paliperidone esters such as paliperidone palmitate dissolve slowly after an i.m. injection before being hydrolyzed to paliperidone and made available in the systemic circulation.

Many patients with the mental illnesses achieve symptom stability with available oral antipsychotic medications; however, it is estimated that up to 75% have difficulty adhering to a daily oral treatment regimen, i.e. compliance problems. Problems with adherence often result in worsening of symptoms, suboptimal treatment response, frequent relapses and re-hospitalizations, and an inability to benefit from rehabilitative and psychosocial therapies. Paliperidone palmitate injection has been developed to provide sustained plasma concentrations of paliperidone when administered once monthly, which may greatly enhance compliance with dosing. Paliperidone palmitate formulated as an aqueous nanosuspension is described in U.S. Pat. Nos. 6,577,545 and 6,555,544. In addition, a dosing regimen of paliperidone palmitate for treating patients is disclosed in US Patent Application Publication No. 20090163519.

Paliperidone palmitate is an atypical antipsychotic drug administered by injection. Paliperidone palmitate may be administered at flexible injection sites including gluteal or deltoid muscle. Previous oil-based antipsychotic agents are indicated for gluteal muscle injection and may be associated with pain on injection, which may cause undesired effects of needle phobia and perceived injection pain. This may reduce patients' acceptance towards these medications and result in a negative influence on the clinical management of these patients. The administration of paliperidone palmitate at flexible injection sites may improve patients' acceptance and compliance to psychotic treatment.

In addition, paliperidone palmitate provides benefits of sustained dose release in plasma without significant concentration variation, regular monitor, reduced side effects and increased treatment efficacy. The administration of paliperidone palmitate may improve effectiveness of psychotic treatment.

Therefore, there may be an increasing demand to switch treatment of patients in need thereof from oral or injectable antipsychotic drugs to paliperidone palmitate. Further, there is a need to reinitiate a dosing regimen for patients who misses their maintenance or stabilized dose. Thus, the objective of the present application is to provide a dosing regimen of paliperidone palmitate for patients in need of a treatment switching from other antipsychotic agents to paliperidone palmitate. Another objective of the present application is to provide a dosing regimen of paliperidone palmitate for patients who have missed the monthly maintenance or stabilized dosing regimen of paliperidone palmitate.

### SUMMARY OF THE INVENTION

In one embodiment of the present application a dosing regimen is provided for administering paliperidone palmitate to a patient in need of psychiatric treatment, wherein said patient misses a stabilized monthly maintenance dose for more than about 4 weeks and less than about 6 weeks, comprising administering intramuscularly in the deltoid a first reinitiation loading dose of paliperidone as a paliperidone palmitate formulated in a sustained release formulation on the first day of treatment; and administering intramuscularly in the gluteal a reinitiation maintenance dose of paliperidone as a paliperidone ester in a sustained release formulation on the 23^{rd} day to about the 37^{th} day or between about 30±7 day after said first day of treatment.

In another embodiment of the present application a dosing regimen is provided for administering paliperidone esters to a patient in need of psychiatric treatment, wherein said patient misses a stabilized monthly maintenance dose for more than about 6 weeks, comprising administering intramuscularly in the deltoid a first reinitiation loading dose of paliperidone as a paliperidone palmitate formulated in a sustained release formulation on the first day of treatment; administering intramuscularly in the deltoid a second reinitiation loading dose of paliperidone as a paliperidone palmitate formulated in a sustained release formulation 1 week later (on the eighth day of treatment); and administering intramuscularly in the gluteal a reinitiation maintenance dose of paliperidone as a paliperidone ester in a sustained release formulation on about the 23^{rd} day to about the 37^{th} day or between about 30±7 days after said first day of treatment.

According to the present application, the first reinitiation dose and the second reinitiaiton dose may be the same dosing as the stabilized monthly maintenance dose. Further, the first reinitiation dose, the second reinitiation dose and the reinitiation maintenance dose of paliperidone as a paliperidone palmitate formulated in a sustained release formulation may range from about 39 mg to about 234 mg.

In yet another embodiment of the present application a dosing regimen is provided for administering paliperidone palmitate to a psychiatric patient in need of a switching treatment to paliperidone palmitate, wherein said patient has received injectable antipsychotic drugs other than paliperidone palmitate, comprising administering intramuscularly in the deltoid of said patient a first loading dose of paliperidone as a paliperidone palmitate formulated in a sustained release formulation on the first day of treatment; and administering intramuscularly in the deltoid or gluteal muscle of said patient a maintenance dose of paliperidone palmitate in a sustained release formulation on about the 23^{rd} day to about the 37^{th} day or between about 30±7 days after said first day of treatment.

In a further embodiment of the present application a dosing regimen is provided for administering paliperidone palmitate to a psychiatric patient in need of a switching treatment to paliperidone palmitate, wherein said patient has received injectable antipsychotic drugs other than paliperidone palmitate, comprising administering intramuscularly in the deltoid of said patient a first loading dose of paliperidone as a paliperidone palmitate formulated in a sustained release formulation on the first day of treatment; administering intramuscularly in the deltoid or gluteal muscle of said patient a maintenance dose of paliperidone palmitate in a sustained release formulation on about the 23^{rd} day to about the 37^{th} day or between about 30±7 days after said first day of treatment; and administering in the deltoid or gluteal muscle of said patient said maintenance dose of paliperidone palmitate in a sustained release formulation monthly thereafter.

According to the present application, the first dose and the maintenance dose of paliperidone for the switch treatment as a paliperidone palmitate formulated in a sustained release formulation may range from about 39 mg to about 234 mg.

Further according to the present application, the first dose and the maintenance dose of paliperidone for the switch treatment as a paliperidone palmitate formulated in a sustained release formulation may range from about 39 mg to about 234 mg.

This and other objects and advantages of the present invention may be appreciated from a review of the present applications.

### DETAILED DESCRIPTION OF FIGURES

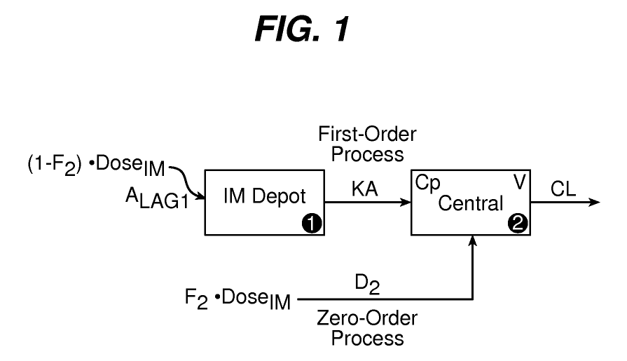

FIG. 1. Diagram of the final model for paliperidone palmitate.

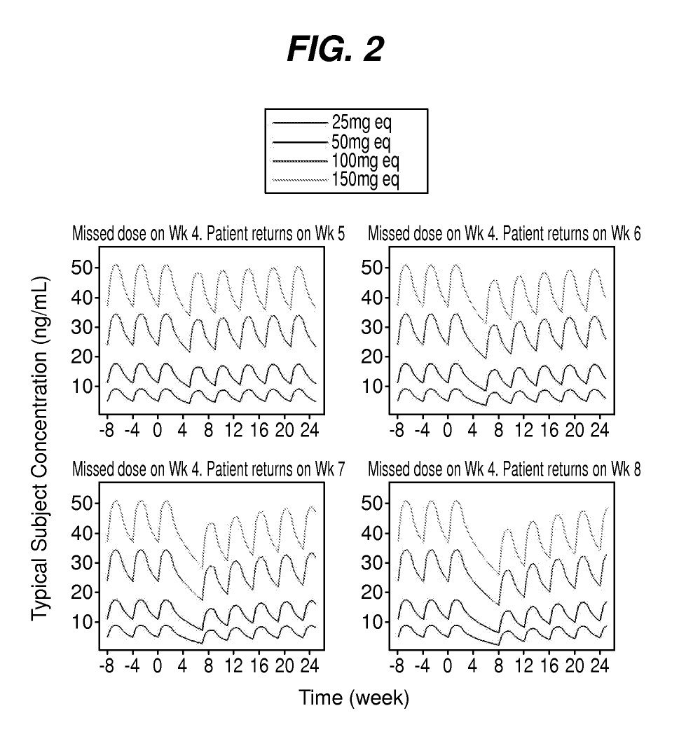

FIG. 2. Simulations for reinitiation treatment of patients who missed the week 4 dose at about weeks 5, 6, 7, and 8 with a single maintenance dose of at day 1.

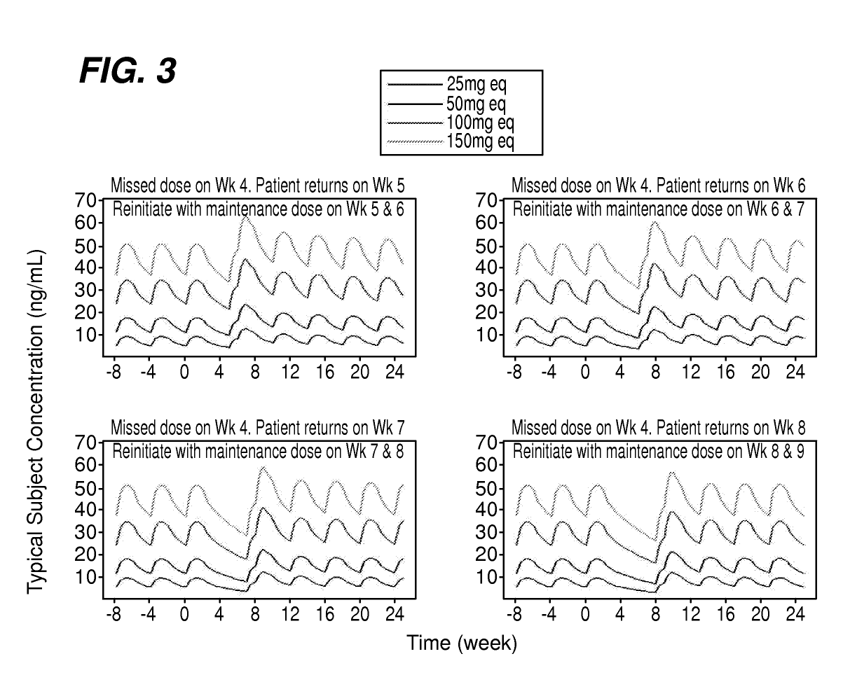

FIG. 3. Simulation of reinitiation treatment of patients who missed the week 4 dose at about weeks 5, 6, 7, and 8 with two maintenance doses at day 1/day 8.

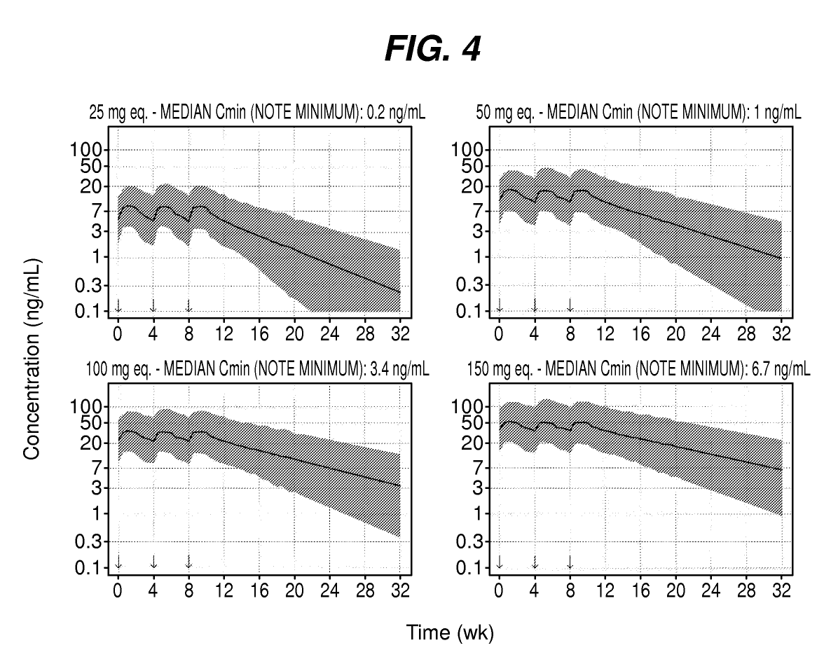

FIG. 4: Plasma concentration profiles of steady-state paliperidone palmitate following more than about 6 months of treatment lapse, using various doses of paliperidone palmitate.

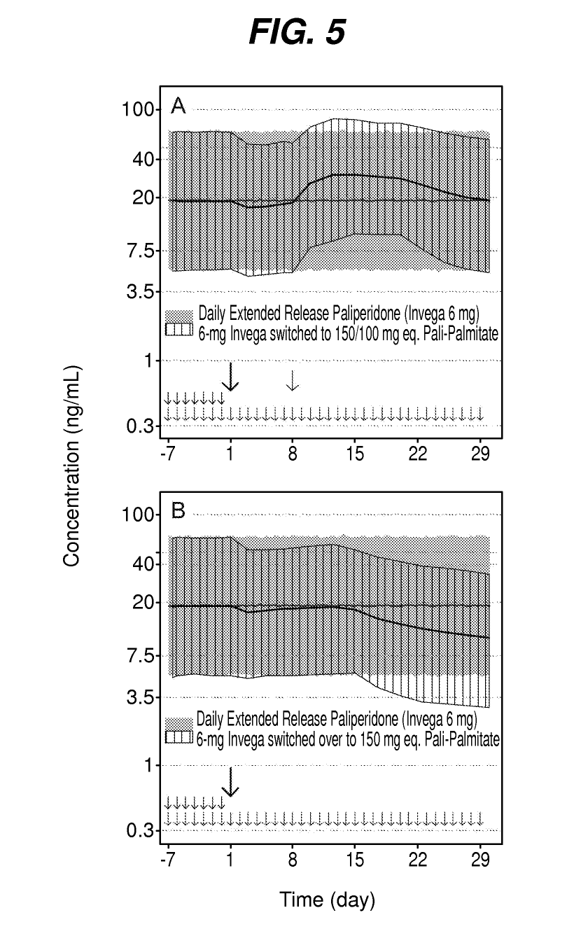

FIG. 5. Switching treatment from oral paliperidone ER to paliperidone palmitate. Pink shaded areas represent patients stabilized on oral ER paliperidone and continuing oral therapy. (A) Hatched area represents patients switched to paliperidone palmitate on day 1 using the day1/day8 initiation. (B) Hatched area represents patients switched to paliperidone palmitate on day 1 using a single initiation dose alone. Lines & shaded/hatched areas represent median and about 90% prediction intervals; arrows indicate dosing times.

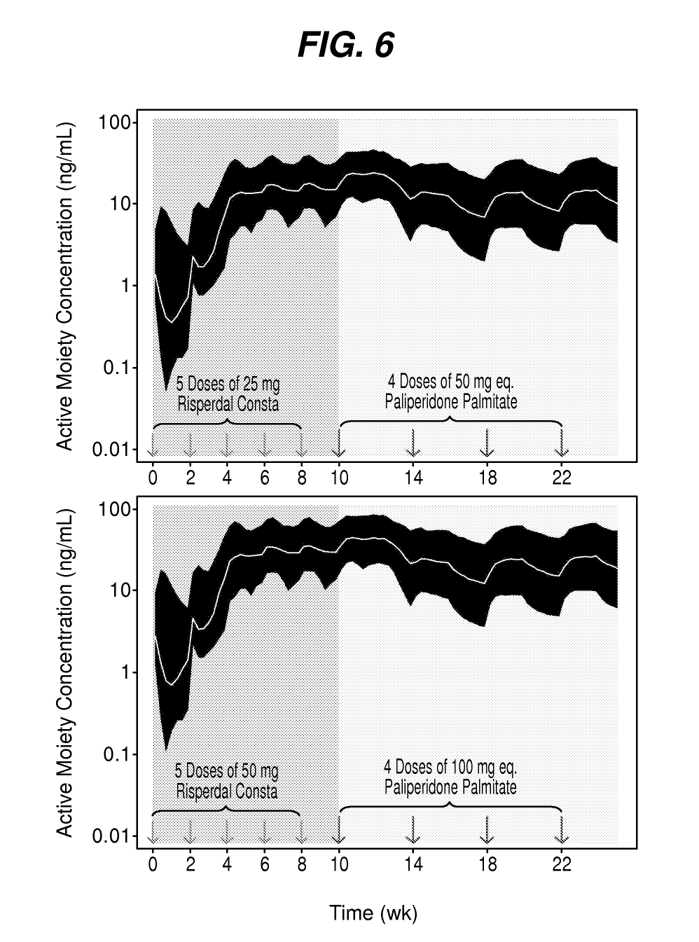

FIG. 6. Switching from RISPERDAL® CONSTA® to paliperidone palmitate. Top panel represents the low dose and the bottom panel represents the high dose. Simulations for the middle dose are not shown because those results can be simply interpolated between the 2 panels. Lines and shaded areas (violet region) represent medians and about 90% prediction intervals.

### DETAILED DESCRIPTION

In one aspect the present application provides a dosing regimen for paliperidone palmitate comprising administering a initial dosing at the first day of treatment and administering a maintenance dosing on between 30±7 days after the first day of treatment.

Paliperidone palmitate is a long-acting intramuscular injectable atypical antipsychotic. Paliperidone palmitate is an ester of paliperidone which has been approved in the US and other countries for the acute and maintenance treatment of patients with schizophrenia. Following intramuscular injection, paliperidone is released into the systemic circulation over an extended period of time, allowing for once-monthly dosing without the need for oral supplementation.

U.S. Patent Application No. 20090163519 has disclosed a dosing regimen for treating a psychiatric patient using paliperidone as a paliperidone palmitate ester in a sustained release formulation. To attain a therapeutic plasma level of paliperidone, patients are administered to receive a first dose of paliperidone palmitate on day 1 of treatment, followed by a second dose between days 6 to 10 of treatment, then a third dose between days 34 to 38 of treatment. It is preferred that the patients will be administered the first dose on day 1, the second dose on day 8 after the first dose and the third dose on day 36 of after the first dose. The first two doses may be injected in the deltoid muscle. Thereafter paliperidone palmitate may be administered by injection approximately once a month (e.g. once every four weeks). To assure a potential therapeutic plasma level of paliperidone is attained, at least the first loading dose of about 150 mg-eq. of paliperidone as a paliperidone palmitate ester may be administered on day 1 of treatment. To further assure a potential therapeutic plasma level of paliperidone is attained by the patient, the first loading dose and the second loading dose ranging between from about 100 mg-eq. to about 150 mg-eq. of paliperidone as a paliperidone palmitate ester may be administered. To maintain a therapeutic level in the plasma, the subsequent doses thereafter or the maintenance dose ranging from about 25 mg-eq. to 150 mg-eq. per month may be administered. The maintenance dose may be administered intramuscularly into the deltoid or gluteal muscle, and the gluteal muscle is preferred. Those of ordinary skill in the art will understand that the maintenance dose may be titrated up or down in view of the patients' conditions such as response to the medication and renal function.

Due to the improved drug efficacy, long-acting sustained release formulation, and reduced side effects of paliperidone palmitate, there may be clinical need and increasing demand to switch patients from previous antipsychotic drugs to paliperidone palmitate.

As described herein, various dosing regimen including switching treatment and reinitiation treatment for paliperidone palmitate is generated from comprehensive pharmacokinetic models or simulations based on clinical data. The models or simulations provide useful, efficient and cost-effective treatment since there is no systematically collected clinical data to specifically address switching schizophrenia patients from other antipsychotics to paliperidone palmitate or concerning concomitant administration with other antipsychotics. Based on the extensive analysis of Phases I, II and III clinical trials with schizophrenia patients, the pharmacokinetic models provide an optimal effective regimen for switching treatment of patients from other antipsychotic drug to paliperidone palmitate and reinitiation treatment of patients missed their stabilized doses of paliperidone palmitate.

The models have indicated that there may be flexibility in the duration of the second loading dose and the maintenance dose of the maintenance dosing regimen. For example, the second loading dose may be administered within the duration of about the 8^{th} day±2 days (or about 1 week±2 days) after administering of the first loading dose. Therefore, the second loading dose may be administered from about the 6^{th} to about the 10^{th} day after the first loading dose of the initial dosing. Similarly, the maintenance dose may be administered within the duration of about the 30^{th} day±7 days after administering of the first loading dose. Therefore, the maintenance dose may be administered from about the 23^{rd} day to about the 37^{th} day after administering of the first loading dose of the initial dosing. The flexible administration timing provides additional treatment benefit for patients who may require earlier administration or have missed their dose, within a short window, of the scheduled treatment without affecting the treatment effectiveness.

The models or simulations also indicate that paliperidone palmitate may be administered by intramuscular injection into either deltoid or gluteal muscle. The first and second loading dose of the initiation regimen may be administered in the deltoid muscle and the maintenance dose of the maintenance regimen may be administered in either the deltoid or gluteal muscle. The injection into the deltoid muscle may be delivered by a 1-inch 23-Gauge (G) or 1.5-inch 22-G needle based on the patient's weight. For the patients whose body weights are less than about 90 kg or 200 lb, a 1-inch 23-G needle may be used for administration, and for those body weights are equal or more than about 90 kg or 200 lb, a 1.5-inch 22-G needle may be used for administration. The injection into the gluteal muscle may be delivered by a 1.5-inch 22-G needle for all body weights.

One aspect of the present application provides a method or dosing regimen for treating patients switching from previous injectable or oral antipsychotic drug to paliperidone palmitate. The previous injectable antipsychotic drug may include but not limited to clopenthixol decanoate, perphenazine enanthate, pipothiazine palmitate, haloperidol decanoate, fluspirilene, zuclopenthixol decanoate, flupenthixol decanoate, fluphenazine decanoate, fluphenazine enanthate, risperidone microspheres, olanzapine pamoate and the like. The previous oral antipsychotic drug may include oral typical antipsychotic such as chlorpromazine, flupenthixol, fluphenazine, haloperidol, loxapine, molindone, perphenazine, pimozide, prochlorperazine, thioridazine, thiothixene, trifluoperazine or the like; and oral atypical antipsychotic drug such as amisulpride, aripiprazole, clozapine, olanzapine, quetiapine, risperidone active moiety, sertindole, ziprasidone and the like.

For patients who have previously received injectable antipsychotic drugs, a switching treatment to paliperidone palmitate may comprise an initiation dosing regimen and a maintenance dosing regimen. The switching treatment may be initiated in place of the next scheduled injection. It is found herein that one dosing of paliperidone palmitate may be sufficient to attain the desired drug levels or plasma concentration of paliperidone during the initial dosing regimen. Accordingly, the initiation dosing regimen for switching patients from other injectable antipsychotic may comprise administering a first loading dose of paliperidone palmitate. Thereafter, the patients may be administered with the maintenance dosing regimen of paliperidone palmitate at a monthly schedule. The maintenance dosing regimen may comprise administering a maintenance dose of paliperidone palmitate on between days 23 to 37 after the first loading dose.

The dose of the switching treatment from previous injectable antipsychotic may be determined based on the condition of the patient and/or the severity of the disease. The preferred first loading dose may range from about 156 mg to about 234 mg of paliperidone palmitate, and more preferably about 234 mg. The preferred monthly maintenance dose may range from about 39 to about 234 mg, and more preferably about 117 mg. Based on the patient tolerability and/or the drug efficacy, the maintenance dose may be further adjusted monthly to achieve optimal treatment effectiveness.

By way of example, a dosing regimen is provided to switch patients from other injectable antipsychotic drug to paliperidone palmitate comprising administering into the deltoid muscle the initial dosing regimen comprising a first loading dose of about 234 mg of paliperidone palmitate and administering into the deltoid or gluteal muscle the maintenance regimen comprising a monthly maintenance dose of about 39 to about 234 mg of paliperidone palmitate on about the 23^{rd} day to about the 37^{th} day after administering of the first loading dose.

For patients who have previously received oral antipsychotic drugs, a switching treatment to paliperidone palmitate may comprise an initial dosing regimen and a monthly dosing regimen. The initial dosing regimen may comprise administering a first loading dose of paliperidone palmitate and administering a second loading dose of paliperidone palmitate, and the maintenance dosing regimen may comprise administering a maintenance dose of paliperidone palmitate. The previous oral antipsychotics may be discontinued at the time of initiation of the switching treatment or administration of the first loading dosing of paliperidone palmitate.

To initiate switching treatment from oral antipsychotic drug, paliperidone palmitate may be initiated with the first loading dose on treatment day 1 and the second loading dose one week later, and maintained with the maintenance dose at a monthly schedule. The dose may be determined based on the condition of the patient and/or the severity of the disease. The preferred first loading dose may range from about 156 mg to about 234 mg of paliperidone palmitate, and more preferably about 234 mg. The preferred second loading dose may range from about 78 mg to about 156 mg, and more preferably about 156 mg. The preferred monthly maintenance dose may range from about 39 to about 234 mg, and more preferably about 117 mg. Subsequently, based on the patient tolerability and/or the drug efficacy, the maintenance dose may be further adjusted monthly to achieve optimal treatment effectiveness. The patients may be monitored for several months to ensure the full effect of the dose adjustment because of the prolonged-release characteristic of paliperidone palmitate.

Based on the pharmacokinetic simulations, patients previously stabilized on paliperidone in oral tablets may attain similar paliperidone steady-state exposure during maintenance treatment with paliperidone palmitate intramuscular injection monthly. For example, patients stabilized on oral paliperidone of about 3 mg may attain similar paliperidone steady-state exposure with the intramuscular injection of paliperidone palmitate of about 39 mg to about 78 mg. Similarly, patients stabilized on oral paliperidone of about 6 mg and about 9 mg may attain similar paliperidone steady-state exposure with the intramuscular injection of paliperidone palmitate of about 117 mg and about 234 mg, respectively. Therefore, during the maintenance regimen, the patients previously stabilized on paliperidone in oral tablets may be administered with the appropriate dose of paliperidone palmitate in injectable formulation corresponding to the stabilized dose of oral paliperidone.

Another aspect of the present application provides a method for treating patients who have missed the stabilized dosing regimen. As generally recommended in the medical field, a missed dose during treatment regimen should be avoided. Because of the flexibility in the duration of the initiation dosing regimen and the maintenance dosing regimen as discussed above, the second loading dose of the initial regimen may be administered at about the 8^{th} day±2 days (1 week±2 days) after administering of the first loading dose. Similarly, the maintenance dose of the maintenance regimen may be administered at about the 30^{th} day±7 days after administering of the first loading dose. This may avoid or reduce the frequency of a missed dose of paliperidone palmitate during the treatment.

Using the pharmacokinetic model or simulation, a dosing regimen is provided for the reinitiation regimen for administering paliperidone palmitate to patients who have missed the monthly maintenance dose by more than about 4 weeks. The reinitiation regimen may depend upon the duration of time lapsed since the last injection of paliperidone palmitate. By way of example, a reinitiation regimen may be provided for treating patients who have missed a dose for more than about 4 weeks and less than about 6 weeks, for more than about 6 weeks and less than about 6 months, and for more than about 6 months.

When more than about 4 weeks and less than about 6 weeks have elapsed since a patient received the last dosing of paliperidone palmitate, the reinitiation regimen may comprise a first loading dose and a maintenance dose. The first dose of may be administered as soon as possible and the maintenance dose may be administered at monthly intervals after the first loading dose. The duration of the maintenance dose may be flexible, e.g. the maintenance dose may be administered 30 days±7 days or the 23^{rd} day to the 37^{th} day after the first loading dose. It is found herein that the administration of a single dose of paliperidone palmitate at the treatment day 1 provides sufficient drug levels or plasma concentrations of paliperidone. Therefore, a second loading dose at day 8 is not needed for treating the patients who missed stabilized dose for less than about 6 weeks.

The first dose and the maintenance dose may be the same dosing amount as the previously stabilized dose of the maintenance regimen prior to the missed dose. Each of the first and the maintenance doses of the reinitiation regimen for less than about 6 weeks may range from about 39 mg to about 234 mg of paliperidone palmitate. Additionally, the maintenance dosing of the reinitiation regimen for less than about 6 weeks may be injected in either deltoid or gluteal muscle.

In one embodiment, a method of reinitiation regimen is provided for treating patients who have missed a dose for more than about 4 weeks and less than about 6 weeks, comprising administering into the deltoid muscle a first loading dose and administering into the deltoid or gluteal muscle a maintenance dose on about the 23^{rd} to about the 37^{th} day after the first loading dose. Thereafter, the maintenance may be administered into the deltoid or gluteal muscle at a monthly schedule.

When more than 6 weeks and less than about 6 months have elapsed since a patient received the last dosing of paliperidone palmitate, the reinitiation regimen may comprise a first loading dose, a second loading dose, and a maintenance dose. The first dose of may be administered as soon as possible, the second dose may be administered at about the 8^{th} days (or about 1 week) after the first loading dose, and a maintenance dosing may be administered at about the 30^{th} day after the first loading dose. Thereafter, the maintenance dose may be administered at monthly intervals. The duration of the second loading dose and the maintenance dose may be flexible. For example, the second loading dose may be administered 8 days±2 days or the 6^{th} day to the 10^{th} day after the first loading dose and the maintenance dose may be administered 30 day±7 days (or the 23^{rd} day to the 37^{th} day) after the first loading dose. The first dose and the second dose of the reinitiation regimen for more than about 6 weeks and less than 6 months may be injected in deltoid muscle to provide a quick attainment to the desired drug levels or plasma concentrations of paliperidone. The first dose and the second dose may depend on the stabilized dose prior to the missed dose. By way of example, when the stabilized dose prior to the missed dose is less than about 234 mg of paliperidone palmitate, the first loading dose and the second loading dose may be the same dosing amount as the stabilized dose prior to the missed dose. For example, each of the first loading dose and the second loading dose may range from about 39 mg to about 156 mg of paliperidone palmitate. By way of another example, when the stabilized dose prior to the missed dose is about 234 mg of paliperidone palmitate, the first loading may be administered at about 156 mg and the second loading dose may be administered at about 156 mg. Thereafter, the maintenance dosing may range from about 39 mg to about 234 mg of paliperidone palmitate and may be injected in either deltoid or gluteal muscle.

In another embodiment, a method of reinitiation regimen is provided for treating patients who have missed a dose for more than about 6 weeks and less than 6 months, comprising administering into the deltoid muscle a first loading dose, administering into the deltoid muscle a second loading dose on about the 6^{th} day to the 10^{th} day after the first loading dose, and administering into the deltoid or gluteal muscle a maintenance dose on about the 23^{rd} day to the 37^{th} day after the first loading dose. Thereafter, the maintenance dose may be administered at monthly intervals.

When more than about 6 months have elapsed since a patient received the last dosing of paliperidone palmitate, the reinitiation regimen may comprise a first loading dose, a second loading dose and a maintenance dose. The first dose may be administered as soon as possible, the second dose may be administered on about the 8^{th} day after the first loading dose, and a maintenance dosing may be administered on about 30^{th} day after the first loading dose. The duration of the second loading dose and the maintenance dose of the reinitiation regimen may be flexible. For example, the second loading dose may be administered 7th day±2 days or the 6^{th} day to the 10^{th} day after the first loading dose and the maintenance dose may be administered about 30 day±7 days or the 23^{rd} day to the 37^{th} day after the loading dose.

The dose of the reinitiaiton regimen for more than about 6 months may be determined based on the condition of the patient and/or the severity of the disease. The preferred first loading dose may range from about 156 mg to about 234 mg of paliperidone palmitate, and more preferably about 234 mg. The preferred second loading dose may range from about 78 mg to about 156 mg, and more preferably about 156 mg. The preferred monthly maintenance dose may range from about 39 to about 234 mg, and more preferably about 117 mg. Subsequently, based on the patient tolerability and/or the drug efficacy, the maintenance dose may be further adjusted monthly to achieve optimal treatment effectiveness. The patients may be monitored for several months to ensure the full effect of the dose adjustment because of the prolonged-release characteristic of paliperidone palmitate. Further, the first dose and the second dose of the reinitiation regimen for patients who have missed the dose for more than about 6 months may be injected in deltoid muscle. The maintenance dose of the reinitiation regimen for patients who have missed the dose for more than about 6 weeks may be injected in either deltoid or gluteal muscle.

In yet another embodiment, a method of reinitiation regimen is provided for treating patients who have missed a dose for more than about 6 months, comprising administering into the deltoid muscle a first loading dose, administering into the deltoid muscle a second loading dose on about the 6^{th} to about the 10^{th} day and administering into the deltoid or gluteal muscle a maintenance dose on about the 23^{rd} day to about the 37^{th} day after administering of the first loading dose. Thereafter, the maintenance dose may be administered at monthly intervals.

As used herein, the term “stabilized dose” refers to the dose which is to be administered according the established dosing regimen. Preferably, the stabilized dose may the maintenance dose of the monthly maintenance dosing regimen prior to a missed dose.

Also used herein, the terms “the first loading dose of the reinitiation regimen”, “the first dose of the reinitiation regimen”, “the first reinitiation dose” or variant thereof refer to the dose to be administered on day 1 when patients return to treatment. Similarly, the terms “the second loading dose of the reinitiation regimen”, “the second dose of the reinitiation regimen”, “the second reinitiation dose” or variant thereof refer to the dose to be administered after a week after the treatment day 1; and the terms “the maintenance dose of the reinitiation regimen”, “the reinitiation maintenance dose” or variant thereof refer to the dose to be administered monthly after the treatment day 1.

Paliperidone esters are psychotic agents belonging to the chemical class of benzisoxazole derivatives, which contains a racemic mixture of (+)- and (−)-paliperidone, which are described in U.S. Pat. No. 5,254,556 (incorporated herein by reference). The chemical name for paliperidone palmitate is (±)-3-[2-[4-(6-fluoro-1,2-benzisoxazol-3-yl)-1-piperidinyl]ethyl]-6,7,8,9-tetrahydro-2-methyl-4-oxo-4H-pyrido[1,2-c]pyrimidin-9-yl hexadecanoate. The structural formula is:

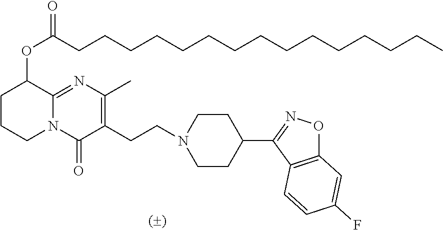

Paliperidone esters may be formulated with pharmaceutical excipients into injectable dosage forms as described in U.S. Pat. Nos. 5,254,556 and 6,077,843 both of which are incorporated herein by reference. Injectable formulations may be formulated in aqueous carriers.

Suitable aqueous depot formulations are described in U.S. Pat. No. 6,077,843 which is incorporated herein by reference. The aqueous formulation would preferably be a nano particle suspension of wherein the nano particles would be of an averages size of less than about 2,000 nm to about 100 nm. Preferably the nano particles would have an average particle size (d50) of from about 1,600 nm to about 400 nm and most preferably about 1,400 nm to about 900 nm. Preferably the d90 will be less than about 5,000 nm and more preferably less than about 4,400 nm. As used herein, an effective average particle size (d50) of less than about 2,000 nm means that at least 50% of the particles have a diameter of less than about 2,000 nm when measured by art-known conventional techniques, such as sedimentation field flow fractionation, photon correlation spectroscopy or disk centrifugation. With reference to the effective average particle size, it is preferred that at least about 90%, e.g. about 5,000 nm. Most preferably, about 90% of the particles have a size of less than about 4,400 nm.

Suitable aqueous nanoparticle depot formulations are described in U.S. Pat. No. 6,555,544 which is incorporated herein by reference. In one embodiment of the present invention the formulation would comprise nanoparticles, a surfactant, a suspending agent, and optionally one or more additional ingredients selected from the group consisting of preservatives, buffers and an isotonizing agent.

Useful surface modifiers are believed to include those that physically adhere to the surface of the active agent but do not chemically bond thereto. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants. Representative examples of excipients include gelatin, casein, lecithin (phosphatides), gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available TWEENS™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phtalate, noncrystalline cellulose, magnesium aluminate silicate, triethanolamine, polyvinyl alcohol (PVA), poloxamers, tyloxapol and polyvinylpyrrolidone (PVP). Most of these excipients are described in detail in the Handbook of Pharmaceutical Excipients, published jointly by the American Pharmaceutical Association and The Pharmaceutical Society of Great Britain, the Pharmaceutical Press, 1986. The surface modifiers are commercially available and/or can be prepared by techniques known in the art. Two or more surface modifiers can be used in combination.

Particularly preferred surface modifiers include polyvinylpyrrolidone; tyloxapol; poloxamers, such as PLURONIC™ F68, F108 and F127 which are block copolymers of ethylene oxide and propylene oxide available from BASF; poloxamines, such as TETRONIC™ 908 (T908) which is a tetrafunctional block copolymer derived from sequential addition of ethylene oxide and propylene oxide to ethylenediamine available from BASF; dextran; lecithin; Aerosol OT™ (AOT) which is a dioctyl ester of sodium sulfosuccinic acid available from Cytec Industries; DUPONOL™ P which is a sodium lauryl sulfate available from DuPont; TRITON™ X-200 which is an alkyl aryl polyether sulfonate available from Rohm and Haas; TWEEN™ 20, 40, 60 and 80 which are polyoxyethylene sorbitan fatty acid esters available from ICI Speciality Chemicals; SPAN™ 20, 40, 60 and 80 which are sorbitan esters of fatty acids; ARLACEL™ 20, 40, 60 and 80 which are sorbitan esters of fatty acids available from Hercules, Inc.; CARBOWAX™ 3550 and 934 which are polyethylene glycols available from Union Carbide; CRODESTA™ F110 which is a mixture of sucrose stearate and sucrose distearate available from Croda Inc.; CRODESTA™ SL-40 which is available from Croda, Inc.; hexyldecyl trimethyl ammonium chloride (CTAC); bovine serum albumin and SA90HCO which is C₁₈H₁₇CH₂(CON(CH₃)CH₂(CHOH)₄CH₂OH)₂. The surface modifiers which have been found to be particularly useful include tyloxapol and a poloxamer, preferably, Pluronic™ F108 and Pluronic™ F68.

Pluronic™ F108 corresponds to poloxamer 338 and is the polyoxyethylene, polyoxypropylene block copolymer that conforms generally to the formula HO[CH₂CH₂O]ₓ[CH(CH₃)CH₂O]_{y}[CH₂CH₂O]_{z}H in which the average values of x, y and z are respectively 128, 54 and 128. Other commercial names of poloxamer 338 are Hodag NONIONIC™ 1108-F available from Hodag, and SYNPERONIC™ PE/F108 available from ICI Americas.

The optimal relative amount of paliperidone palmitate and the surface modifier depends on various parameters. The optimal amount of the surface modifier can depend, for example, upon the particular surface modifier selected, the critical micelle concentration of the surface modifier if it forms micelles, the surface area of the antipsychotic agent, etc. The specific surface modifier preferably is present in an amount of about 0.1 to about 1 mg per square meter surface area of the paliperidone palmitate. It is preferred in the case of paliperidone palmitate (9-hydroxyrisperidone palmitate) to use PLURONIC™ F108 as a surface modifier, a relative amount (w/w) of both ingredients of approximately 6:1 is preferred.

The particles of this invention can be prepared by a method comprising the steps of dispersing paliperidone palmitate in a liquid dispersion medium and applying mechanical means in the presence of grinding media to reduce the particle size of the antipsychotic agent to an effective average particle size of less than about 2,000 nm. The particles can be reduced in size in the presence of a surface modifier. Alternatively, the particles can be contacted with a surface modifier after attrition.

A general procedure for preparing the particles of this invention includes (a) obtaining paliperidone palmitate in micronized form; (b) adding the micronized paliperidone palmitate to a liquid medium to form a premix; and (c) subjecting the premix to mechanical means in the presence of a grinding medium to reduce the effective average particle size.

The paliperidone palmitate in micronized form may be prepared using techniques known in the art. It is preferred that the particle size of the micronized paliperidone palmitate be less than about 100 μm as determined by sieve analysis. If the particle size of the micronized paliperidone palmitate is greater than about 100 μm, then it is preferred that the particles of paliperidone palmitate be reduced in size to less than 100 μm.

The micronized paliperidone palmitate can then be added to a liquid medium in which it is essentially insoluble to form a premix. The concentration of paliperidone palmitate in the liquid medium (weight by weight percentage) can vary widely and depends on the selected antipsychotic agent, the selected surface modifier and other factors. Suitable concentrations of paliperidone palmitate in compositions vary from about 0.1% to about 60%, preferably is from about 0.5% to about 30%, and more preferably, is approximately 7% (w/v). It is currently preferred to use a concentration of about 100 mg eq of paliperidone per ml or about 156 mg of paliperidone palmitate per ml.

A more preferred procedure involves the addition of a surface modifier to the premix prior to its subjection to mechanical means to reduce the effective average particle size. The concentration of the surface modifier (weight by weight percentage) can vary from about 0.1% to about 90%, preferably from about 0.5% to about 80%, and more preferably is approximately 7% (w/v).

The premix can be used directly by subjecting it to mechanical means to reduce the effective average particle size in the dispersion to less than about 2,000 nm. It is preferred that the premix be used directly when a ball mill is used for attrition. Alternatively, the antipsychotic agent and, optionally, the surface modifier, can be dispersed in the liquid medium using suitable agitation such as, for example, a roller mill or a Cowles type mixer, until a homogeneous dispersion is achieved.

The mechanical means applied to reduce the effective average particle size of the antipsychotic conveniently can take the form of a dispersion mill. Suitable dispersion mills include a ball mill, an attritor mill, a vibratory mill, a planetary mill, media mills—such as a sand mill and a bead mill. A media mill is preferred due to the relatively shorter milling time required to provide the desired reduction in particle size. For media milling, the apparent viscosity of the premix preferably is anywhere between about 0.1 Pa·s and about 1 Pa·s. For ball milling, the apparent viscosity of the premix preferably is anywhere between about 1 mPa·s and about 100 mPa·s.

The grinding media for the particle size reduction step can be selected from rigid media preferably spherical or particulate in form having an average size less than about 3 mm and, more preferably, less than about 1 mm. Such media desirably can provide the particles of the invention with shorter processing times and impart less wear to the milling equipment. The selection of the material for the grinding media is believed not to be critical. However, about 95% ZrO stabilized with magnesia, zirconium silicate, and glass grinding media provide particles which are acceptable for the preparation of pharmaceutical compositions. Further, other media, such as polymeric beads, stainless steel, titania, alumina and about 95% ZrO stabilized with yttrium, are useful. Preferred grinding media have a density greater than about 2.5 g/cm³ and include about 95% ZrO stabilized with magnesia and polymeric beads.

The attrition time can vary widely and depends primarily upon the particular mechanical means and processing conditions selected. For rolling mills, processing times of up to two days or longer may be required.

The particles must be reduced in size at a temperature which does not significantly degrade the antipsychotic agent. Processing temperatures of less than about 30° C. to about 40° C. are ordinarily preferred. If desired, the processing equipment may be cooled with conventional cooling equipment. The method is conveniently carried out under conditions of ambient temperature and at processing pressures which are safe and effective for the milling process.

The surface modifier, if it was not present in the premix, must be added to the dispersion after attrition in an amount as described for the premix above. Thereafter, the dispersion can be mixed by, for example, shaking vigorously. Optionally, the dispersion can be subjected to a sonication step using, for example, an ultrasonic power supply.

Aqueous compositions according to the present invention conveniently further comprise a suspending agent and a buffer, and optionally one or more of a preservative and an isotonizing agent. Particular ingredients may function as two or more of these agents simultaneously, e.g. behave like a preservative and a buffer, or behave like a buffer and an isotonizing agent.

Suitable suspending agents for use in the aqueous suspensions according to the present invention are cellulose derivatives, e.g. methyl cellulose, sodium carboxymethyl cellulose and hydroxypropyl methyl cellulose, polyvinylpyrrolidone, alginates, chitosan, dextrans, gelatin, polyethylene glycols, polyoxyethylene- and polyoxy-propylene ethers. Preferably sodium carboxymethyl cellulose is used in a concentration of about 0.5 to about 2%, most preferably about 1% (w/v). Suitable wetting agents for use in the aqueous suspensions according to the present invention are polyoxyethylene derivatives of sorbitan esters, e.g. polysorbate 20 and polysorbate 80, lecithin, polyoxyethylene- and polyoxypropylene ethers, sodium deoxycholate. Preferably polysorbate 20 is used in a concentration of about 0.5% to about 3%, more preferably about 0.5% to about 2%, most preferably about 1.1% (w/v).

Suitable buffering agents are salt of weak acids and should be used in amount sufficient to render the dispersion neutral to very slightly basic (up to the pH value of about 8.5), preferably in the pH range of about 7 to about 7.5. Particularly preferred is the use of a mixture of disodium hydrogen phosphate (anhydrous) (typically about 0.9% (w/v)) and sodium dihydrogen phosphate monohydrate (typically about 0.6% (w/v)). This buffer also renders the dispersion isotonic and, in addition, less prone to flocculation of the ester suspended therein.

Preservatives are antimicrobials and anti-oxidants which can be selected from the group consisting of benzoic acid, benzyl alcohol, butylated hydroxyanisole, butylated hydroxytoluene, chlorbutol, a gallate, a hydroxybenzoate, EDTA, phenol, chlorocresol, metacresol, benzethonium chloride, myristyl-gamma-piccolinium chloride, phenylmercuric acetate and thimerosal. In particular, it is benzyl alcohol which can be used in a concentration up to about 2% (w/v), preferably up to about 1.5% (w/v).

Isotonizing agents are, for example, sodium chloride, dextrose, mannitol, sorbitol, lactose, sodium sulfate. The suspensions conveniently comprise from about 0% to about 10% (w/v) isotonizing agent. Mannitol may be used in a concentration from about 0% to about 7% more preferably, however, from about 1% to about 3% (w/v), especially from about 1.5% to about 2% (w/v) of one or more electrolytes are used to render the suspension isotonic, apparently because ions help to prevent flocculation of the suspended ester. In particular, electrolytes of the buffer serve as isotonizing agent.

A particularly desirable feature for an injectable depot formulation relates to the ease with which it can be administered. In particular such an injection should be feasible using a needle as fine as possible in a span of time which is as short as possible. This can be accomplished with the aqueous suspensions of the present invention by keeping the viscosity below about 75 mPa·s, preferably below about 60 mPa·s. Aqueous suspensions of such viscosity or lower can both easily be taken up in a syringe (e.g. from a vial), and injected through a fine needle (e.g a 21 G 1½ inch, 22 G 2 inch, 22 G 1¼ inch or 23 G 1 inch needle). The preferred needles for injection are 22 G 22 G 1½ inch regular wall and 23 G 1 inch regular wall needles.

Ideally, aqueous suspensions according to the present invention will comprise as much prodrug as can be tolerated so as to keep the injected volume to a minimum, and as little of the other ingredients as possible. In particular, such a composition will comprise by weight based on the total volume of the composition: (a) from about 3% to 20% (w/v) of the prodrug; (b) from about 0.5% to 2% (w/v) of a wetting agent; (c) one or more buffering agents sufficient to render the composition neutral to very slightly basic (pH 8.5); (d) from about 0.5% to about 2% (w/v) of a suspending agent; (e) up to about 2% (w/v) preservatives; and (f) water q.s. ad 100%. Preferably the aqueous suspension will be made under sterile conditions and no preservatives will be used. Appropriate methods to aseptically prepare paliperidone palmitate are described in WO 2006/114384 which is hereby incorporated by reference herein.

The preferred aqueous dosage form contains inactive ingredients that are polysorbate 20, polyethylene glycol 4000, citric acid monohydrate, disodium hydrogen phosphate anhydrous, sodium dihydrogen phosphate monohydrate, sodium hydroxide, and water for injection. The mg of compound delivered in such a dosage form to the patient may be from about 25 to about 150 mg (e.g. 25 mg, 50 mg, 75 mg, 100 mg, 150 mg) injectable dosage form.

As used herein, a dose or dosing is expressed as milligrams (mg) of paliperidone palmitate. Paliperidone palmitate dosing may also be expressed as mg equivalents (mg eq.) of paliperidone with about 39, 78, 117, 156, and 234 mg of paliperidone palmitate being equivalent to about 25, 50, 75, 100 and 150 mg eq., of paliperidone, respectively.

The term “antipsychotics” or “antipsychotic drug medication” as used herein means any medication used to decrease or ameliorate the symptoms of psychosis in a person with a psychotic disorder and includes, but is not limited to the following compounds: Acetophenazine Maleate; Alentemol Hydrobromide; Alpertine; Azaperone; Batelapine Maleate; Benperidol; Benzindopyrine Hydrochloride; Brofoxine; Bromperidol; Bromperidol Decanoate; Butaclamol Hydrochloride; Butaperazine; Butaperazine Maleate; Carphenazine Maleate; Carvotroline Hydrochloride; Chlorpromazine; Chlorpromazine Hydrochloride; Chlorprothixene; Cinperene; Cintriamide; Clomacran Phosphate; Clopenthixol; Clopimozide; Clopipazan Mesylate; Cloroperone Hydrochloride; Clothiapine; Clothixamide Maleate; Clozapine; Cyclophenazine Hydrochloride; properidol; Etazolate Hydrochloride; Fenimide; Flucindole; Flumezapine; Fluphenazine Decanoate; Fluphenazine Enanthate; Fluphenazine Hydrochloride; Fluspiperone; Fluspirilene; Flutroline; Gevotroline Hydrochloride; Halopemide; Haloperidol; Haloperidol Decanoate; Iloperidone; Imidoline Hydrochloride; Lenperone; Mazapertine Succinate; Mesoridazine; Mesoridazine Besylate; Metiapine; Milenperone; Milipertine; Molindone Hydrochloride; Naranol Hydrochloride; Neflumozide Hydrochloride; Ocaperidone; Olanzapine; Oxiperomide; Penfluridol; Pentiapine Maleate; Perphenazine; Pimozide; Pinoxepin Hydrochloride; Pipamperone; Piperacetazine; Pipotiazine Palmitate; Piquindone Hydrochloride; Prochlorperazine Edisylate; Prochlorperazine Maleate; Promazine Hydrochloride; Quetiapine; Remoxipride; Remoxipride Hydrochloride; Risperidone; Rimcazole Hydrochloride; Seperidol Hydrochloride; Sertindole; Setoperone; Spiperone; Thioridazine; Thioridazine Hydrochloride; Thiothixene; Thiothixene Hydrochloride; Tioperidone Hydrochloride; Tiospirone Hydrochloride; Trifluoperazine Hydrochloride; Trifluperidol; Triflupromazine; Triflupromazine Hydrochloride; and Ziprasidone Hydrochloride.

The term “psychiatric patient” as used herein, refers to a human, who has been the object of treatment, or experiment for a “mental disorder” and “mental illness” refer to those provided in the Diagnostic and Statistical Manual (DSM IV), American Psychological Association (APA). Those of ordinary skill in the art will appreciate that paliperidone esters (e.g. paliperidone palmitate) can be administered to psychiatric patients for all the known uses of risperidone. These mental disorders include, but are not limited to, schizophrenia; bipolar disorder or other disease states in which psychosis, aggressive behavior, anxiety or depression is evidenced. Schizophrenia refers to conditions characterized as schizophrenia, schizoaffective disorder and schizophreniform disorders, in DSM-IV-TR such as category 295.xx. Bipolar Disorder refers to a condition characterized as a Bipolar Disorder, in DSM-IV-TR such as category 296.xx including Bipolar I and Bipolar Disorder II. The DSM-IV-TR was prepared by the Task Force on Nomenclature and Statistics of the American Psychiatric Association, and provides clear descriptions of diagnostic categories. Pathologic psychological conditions, which are psychoses or may be associated with psychotic features include, but are not limited to the following disorders that have been characterized in the DSM-IV-TR. Diagnostic and Statistical Manual of Mental Disorders, Revised, 3rd Ed. (1994). The skilled artisan will recognize that there are alternative nomenclatures, nosologies, and classification systems for pathologic psychological conditions and that these systems evolve with medical scientific progress. Examples of pathologic psychological conditions which may be treated include, but are not limited to, Mild Mental Retardation (317), Moderate Mental Retardation (318.0), Severe Mental Retardation (318.1), Profound Mental Retardation (318.2), Mental Retardation Severity Unspecified (319), Autistic Disorders (299.00), Rett's Disorder (299.80), Childhood Disintegrative Disorders (299.10), Asperger's Disorder (299.80), Pervasive Developmental Disorder Not Otherwise Specified (299.80), Attention-Deficit/Hyperactivity Disorder Combined Type (314.01), Attention-Deficit/Hyperactivity Disorder Predominately Inattentive Type (314.00), Attention-Deficit/Hyperactivity Disorder Predominately Hyperactive-Impulsive Type (314.01), Attention-Deficit/Hyperactivity Disorder NOS (314.9), Conduct Disorder (Childhood-Onset and Adolescent Type 312.8), Oppositional Defiant Disorder (313.81), Disruptive Behavior Disorder Not Otherwise Specified (312.9), Solitary Aggressive Type (312.00), Conduct Disorder, Undifferentiated Type (312.90), Tourette's Disorder (307.23), Chronic Motor Or Vocal Tic Disorder (307.22), Transient Tic Disorder (307.21), Tic Disorder NOS (307.20), Alcohol Intoxication Delirium (291.0), Alcohol Withdrawal Delirium (291.0), Alcohol-Induced Persisting Dementia (291.2), Alcohol-Induced Psychotic Disorder with Delusions (291.5), Alcohol-Induced Psychotic Disorder with Hallucinations (291.3), Amphetamine or Similarly Acting Sympathomimetic Intoxication (292.89), Amphetamine or Similarly Acting Sympathomimetic Delirium (292.81), Amphetamine or Similarly Acting Sympathomimetic Induced Psychotic with Delusions (292.11), Amphetamine or Similarly Acting Sympathomimetic Induced Psychotic with Hallucinations (292.12), Cannabis-Induced Psychotic Disorder with Delusions (292.11), Cannabis-Induced Psychotic Disorder with Hallucinations (292.12), Cocaine Intoxication (292.89), Cocaine Intoxication Delirium (292.81), Cocaine-Induced Psychotic Disorder with Delusions (292.11), Cocaine-Induced Psychotic Disorder with Hallucinations (292.12), Hallucinogen Intoxication (292.89), Hallucinogen Intoxication Delirium (292.81), Hallucinogen-Induced Psychotic disorder with Delusions (292.11), Hallucinogen-Induced Psychotic disorder with Delusions (292.12), Hallucinogen-Induced Mood Disorder (292.84), Hallucinogen-Induced Anxiety Disorder (292.89), Hallucinogen-Related Disorder Not Otherwise Specified (292.9), Inhalant Intoxication (292.89), Inhalant Intoxication Delirium (292.81), Inhalant-Induced Persisting Dementia (292.82), Inhalant-Induced Psychotic Disorder with Delusions (292.11), Inhalant-Induced Psychotic with Hallucinations (292.12), Inhalant-Induced Mood Disorder (292.89), Inhalant-Induced Anxiety Disorder (292.89), Inhalant-Related Disorder Not Otherwise Specified (292.9), Opioid Intoxication Delirium (292.81), Opioid-Induced Psychotic Disorder with Delusions (292.11), Opioid Intoxication Delirium (292.81), Opioid-Induced Psychotic Disorder with Hallucinations (292.12), Opioid-Induced Mood Disorder (292.84), Phencyclidine (PCP) or Similarly Acting Arylcyclohexylamine Intoxication (292.89), Phencyclidine (PCP) or Similarly Acting Arylcyclohexylamine Intoxication Delirium (292.81), Phencyclidine (PCP) or Similarly Acting Arylcyclohexylamine Induced Psychotic Disorder with Delusions (292.11), Phencyclidine (PCP) or Similarly Acting Arylcyclohexylamine Induced Psychotic Disorder with Hallucinations (292.12), Phencyclidine (PCP) or Similarly Acting Arylcyclohexylamine Mood Disorder (292.84), Phencyclidine (PCP) or Similarly Acting Arylcyclohexylamine Induced Anxiety Disorder (292.89), Phencyclidine (PCP) or Similarly Acting Arylcyclohexylamine Related Disorder Not Otherwise Specified (292.9), Sedative, Hypnotic or Anxiolytic Intoxication (292.89), Sedation, Hypnotic or Anxiolytic Intoxication Delirium (292.81), Sedation, Hypnotic or Anxiolytic Withdrawal Delirium (292.81), Sedation, Hypnotic or Anxiolytic Induced Persisting Dementia (292.82), Sedation, Hypnotic or Anxiolytic-Induced Psychotic Disorder with Delusions (292.11), Sedation, Hypnotic or Anxiolytic-Induced Psychotic Disorder with Hallucinations (292.12), Sedation, Hypnotic or Anxiolytic-Induced Mood Disorder (292.84), Sedation, Hypnotic or Anxiolytic-Induced Anxiety Disorder (292.89), Other (or Unknown) Substance Intoxication (292.89), Other (or Unknown) Substance-Induced Delirium (292.81), Other (or Unknown) Substance-Induced Persisting Dementia (292.82), Other (or Unknown) Substance-Induced Psychotic Disorder with Delusions (292.11), Other (or Unknown) Substance-Induced Psychotic Disorder with Hallucinations (292.12), Other (or Unknown) Substance-Induced Mood Disorder (292.84), Other (or Unknown) Substance-Induced Anxiety Disorder (292.89), Other (or Unknown) Substance Disorder Not Otherwise Specified (292.9), Obsessive Compulsive Disorder (300.3), Post-traumatic Stress Disorder (309.81), Generalized Anxiety Disorder (300.02), Anxiety Disorder Not Otherwise Specified (300.00), Body Dysmorphic Disorder (300.7), Hypochondriasis (or Hypochondriacal Neurosis) (300.7), Somatization Disorder (300.81), Undifferentiated Somatoform Disorder (300.81), Somatoform Disorder Not Otherwise Specified (300.81), Intermittent Explosive Disorder (312.34), Kleptomania (312.32), Pathological Gambling (312.31), Pyromania (312.33), Trichotillomania (312.39), and Impulse Control Disorder NOS (312.30), Schizophrenia, Paranoid Type, (295.30), Schizophrenia, Disorganized (295.10), Schizophrenia, Catatonic Type, (295.20), Schizophrenia, Undifferentiated Type (295.90), Schizophrenia, Residual Type (295.60), Schizophreniform Disorder (295.40), Schizoaffective Disorder (295.70), Delusional Disorder (297.1), Brief Psychotic Disorder (298.8), Shared Psychotic Disorder (297.3), Psychotic Disorder Due to a General Medical Condition with Delusions (293.81), Psychotic Disorder Due to a General Medical Condition with Hallucinations (293.82), Psychotic Disorders Not Otherwise Specified (298.9), Major Depression, Single Episode, Severe, without Psychotic Features (296.23), Major Depression, Recurrent, Severe, without Psychotic Features (296.33), Bipolar Disorder, Mixed, Severe, without Psychotic Features (296.63), Bipolar Disorder, Mixed, Severe, with Psychotic Features (296.64), Bipolar Disorder, Manic, Severe, without Psychotic Features (296.43), Bipolar Disorder, Manic, Severe, with Psychotic Features (296.44), Bipolar Disorder, Depressed, Severe, without Psychotic Features (296.53), Bipolar Disorder, Depressed, Severe, with Psychotic Features (296.54), Bipolar II Disorder (296.89), Bipolar Disorder Not Otherwise Specified (296.80), Personality Disorders, Paranoid (301.0), Personality Disorders, Schizoid (301.20), Personality Disorders, Schizotypal (301.22), Personality Disorders, Antisocial (301.7), and Personality Disorders, Borderline (301.83). The numbers in parenthesis refer to the DSM-IV-TR categories.

The term “therapeutically effective amount” as used herein, means that amount of active compound or pharmaceutical agent that elicits the biological or medicinal response in human that is being sought by a researcher, medical doctor or other clinician, which includes alleviation of the symptoms of the disease or disorder being treated.

Those of skill in the treatment of diseases could easily determine the effective amount of paliperidone to administer for the treatment of the diseases listed above. By way of example, an effective amount of paliperidone for the treatment of mental disorders would be from about 0.01 mg/kg to about 2 mg/kg body weight. For the present invention it is preferred to dose patients with about 25 mg-eq. to about 150 mg eq. paliperidone or about 39 mg to about 234 mg paliperidone palmitate. The amount of paliperidone palmitate is provided in sufficient amount to provide the equivalent dose of paliperidone after the palmitic acid moiety is removed from the ester (e.g. 156 mg corresponds to paliperidone 100 mg). In one embodiment of present invention wherein paliperidone palmitate is administered by intramuscular injection once per month is preferred.

When asked, approximately half of patients in a 13-week study stated that they preferred deltoid to gluteal injections, with the most common reasons for this preference being that it was easier, less embarrassing and faster than an injection in the gluteal muscle. Moreover, it may be beneficial for patients who favour only deltoid injections due to paranoia and other psychiatric symptomatology. When dosing frequency, aqueous-based formulation and flexibility of injection site to accommodate patients' preference are considered in combination, paliperidone palmitate may provide the advantages of improved convenience and acceptability compared with previous antipsychotic medications. With the availability of paliperidone palmitate, the clinicians may need to manage patients switching treatment from other antipsychotic drugs to paliperidone palmitate.

The following non-limiting examples are provided to further illustrate the present invention.

### Example 1

### Methodology

### Population Pharmacokinetics Models

A comprehensive population pharmacokinetics (PK) model was developed for paliperidone palmitate based on data from previous studies of subjects with schizophrenia. Briefly, a 1-compartment model with first-order elimination best described the PK of paliperidone following intramuscular administration of the paliperidone palmitate ester. As shown in FIG. 1, the absorption component of the model allowed a fraction (F2) of the dose to enter the central compartment relatively quickly via a zero-order process with duration D2. After a certain lag-time, the remaining fraction (1−F2) entered the systemic circulation via a first-order process (KA) that determines the shape of the plasma concentration-time curve following injection. NONMEM® Version V (Icon Development Solutions, Ellicott City, Md.) running with NM-TRAN version III was used to conduct all population PK analyses and simulations in accordance to the NONMEM Users Guides (Icon Development Solutions, Ellicott City, Md.). NONMEM was run using the J&JPRD computational grid using Intel FORTRAN 9.0 compiler for Windows. Generation of data sets for NONMEM simulations and visualization of results were performed using S Plus® 6.0 professional release 2 software (Insightful Corporation, Seattle, Wash.). The model building included pooled data from about 1,795 subjects from six Phase 1 studies and five Phase 2 and 3 studies. A total of 18,530 PK samples with valid concentration time-points were part of the population PK database. The final model from the historical population PK analysis [(Pop PK Report Paliperidone Palmitate)], including all significant subject covariates was used as simulation machinery for assessing various dosing regimens for paliperidone palmitate including missed dose treatment and switching treatment. Additionally, a comprehensive population PK model was developed for the extended release oral formulation of paliperidone or INVEGA. The model was constructed using pooled data from about 1,368 subjects with about 21,183 paliperidone concentrations from all phases of the INVEGA drug development. The PK of paliperidone in plasma was best captured using an open 2-compartment disposition model with linear elimination from the central compartment. The absorption was modeled with a sequential zero-order input into a depot compartment and first-order absorption with a lag-time from the depot to the central compartment. The relatively faster absorption of paliperidone from the oral route allowed identification of the distributive peripheral compartment, which is not discernible in the flip-flopped paliperidone palmitate PK data. The final paliperidone model from this historical analysis, including all significant subject covariates, was used for simulating PK exposure from oral paliperidone at various dose levels.

The PK profiles for about 5,000 subjects were simulated for subjects receiving injectable paliperidone palmitate (INVEGA® SUSTENNA™) and oral paliperidone) (INVEGA®). For each data set, the covariates of interest were obtained by resampling from the subject covariates (resampling unit was the subject) available in the subject PK database for paliperidone palmitate and the joint distribution of subject-specific characteristics was maintained. To evaluate the outcome of the simulations, the population median and about 90% prediction interval of the simulated plasma concentration vs. time profiles were plotted together.

A compartmental model was also developed for RISPERDAL® CONSTA® which included a one-compartment disposition submodel characterized by clearance and volume of distribution and three parallel absorption pathways: an immediate pathway describing the absorption of non-encapsulated risperidone, and a fast and a slow sustained-release pathway. For the model building, data for the RISPERDAL® CONSTA® originating only from the final 20-kg manufacturing scale used in Phase-III trials and “to be marketed” formulation was used as the source information. A two stage approach had to adopted for modeling RISPERDAL® CONSTA® PK because the active moiety profile after intramuscular administration of risperidone depot microsphere formulations was extremely complex (immediate release of a small amount of non-encapsulated risperidone followed by two sustained-release processes differing in the rate of release along with variable delay in release initiation). The model was fitted to individual concentration-time profiles of active moiety. However, the mixed-effects version of the model which included interindividual variability in parameters could not be fitted due to numerical problems with the NONMEM software. Thus, at the first stage, individual estimates of active moiety (risperidone+paliperidone) PK parameters were obtained using clinical studies where intensive blood sampling occurred in about 56 subjects. These estimates were used as part of the second step in a non-parametric approach to perform population simulations. For the simulation data set, the parameters of interest were obtained by resampling the individual estimates (n=5,000 subjects) where the resampling unit was the subject. This method was able to retain the joint distribution of subject-specific parameters. It was also noted that a depiction of inter-subject variability computed using this method would be an underestimate due to the small size that was used in building this model. Therefore, the prediction interval for RISPERDAL® CONSTA® simulations should be interpreted with caution. To evaluate the outcome of the simulations, the population median and about 90% prediction interval of the simulated plasma concentration vs. time profiles were plotted together. Oral supplementation used during the first few weeks of RISPERDAL® CONSTA® therapy is ignored in this modeling to simplify this complex exercise.

To add credence to the simulation exercise for the initiation regimens, model based projections were compared with the limited and/or sparse observed data from clinical studies.

### Example 2

### Missed Doses

To manage patients missed the dose of the treatment, simulations were used to evaluate reinitiation treatment in patients who had missed a week 4 dose of paliperidone palmitate and returned to treatment at weeks 5, 6, 7 or 8. The simulations were also used to evaluate re-initiation treatment in patients who had a prolonged lapse of more than about 6 months. The patient may be administered a single dose at day 1 using the maintenance one that would have been administered at exactly the 4^{th} week, or two doses at day1/day 8 using the same dose as the maintenance dose. Both possibilities were investigated for the about 5, 6, 7, and 8 week scenarios using the doses of about 39, 78, 117, 156, and 234 mg of paliperidone palmitate. The time point at which re-initiation with 2 doses could be appropriate was judged based on visual inspection of simulated curves. The profiles after a missed dose were assessed empirically and proximity to the steady-state levels was the criterion for judging the utility of these dosing schemes.

These results in FIGS. 2 to 4 indicated that the reinitiation treatment after patients missed their Week 4 maintenance dose or the stabilized dose, re-initiation depended upon the time lapse since the last injection. For example, patients who missed their week 4 maintenance dose and returned to re-initiation at week 5 or 6 (i.e., time lapse since last injection is more than about 4 weeks and less than about 6 weeks) may be administered with single re-initiation dose at the previously stabilized dose followed by monthly injections (FIGS. 2 and 3). The doses may be administered in either the deltoid muscle with a 1.0 inch 23-G needle for the patients weighting less than about 90 kg or a 1.5 inch 22-G for those weighting equal or more than about 90 kg, or the gluteal muscle with a 1.5-inch 22-G needle for all weights. Additionally, [FIG. 6, Panel A and B] This is recommended as the models showed that reinitiation with two doses at day 1/day 8 resulted in a higher than desired plasma concentration (FIG. 3).

The simulations also showed that patients who missed their week 4 maintenance dose and returned to re-initiation at week 7 or 8 (i.e., time lapse since last injection is more than about 6 weeks and less than about 6 months) may be administered with two re-initiation doses at the previously stabilized dose followed by monthly injections. The two doses at day 1/day 8 allow re-attainment of steady-state plasma concentration quickly (FIG. 3). Additionally, the two reinitiation doses were injected into the deltoid muscle with a 1.0 inch 23-G needle for the patients weighting less than about 90 kg or a 1.5 inch 22-G for those weighting equal or more than about 90 kg. Each of the two re-initiation doses was the previously stabilized dose, except when the patient was stabilized on a dose of about 234 mg. For the patient stabilized on a dose of about 234 mg of paliperidone palmitate, the model recommended each of the first two doses of about 156 mg of paliperidone palmitate.

The simulations further recommended that patient who missed their week 4 maintenance dose and returned more than about 6 months were required to re-initiate the treatment de novo (FIG. 4). That is, patients were administered with paliperidone palmitate of about 234 mg on day 1 and about 156 mg on day 8. Each dose was administered into the deltoid muscle with needle selection based upon patient weight as discussed above. The re-initiation doses were followed by monthly paliperidone palmitate injections using maintenance dose recommendations as discussed above. Finally, the simulation models indicated that there is a ±2 day dosing window for the administration of the second dose, if needed, and a ±7 day dosing window for the administration of the monthly maintenance doses (data not shown).

### Example 3

### Switch Treatment from Oral Antipsychotic

Pharmacokinetic models or simulations were developed to examine drug levels when patients were switched from extended release (ER) oral paliperidone to paliperidone palmitate. The models also determined whether pervious oral antipsychotics such as paliperidone ER could be discontinued at the time of initiation of treatment with paliperidone palmitate.

The models examined patients who were treated with a daily dosing of about 6 mg paliperidone ER and initiated with paliperidone palmitate on the first day after the last oral dose of paliperidone ER. The simulated concentrations of paliperidone from its palmitate ester were added to the drug levels from paliperidone ER using the superposition principles. The simulation models analyzed two scenarios: (A) patients switched from the dose of about 6 mg paliperidone ER to paliperidone palmitate using the two initiation doses of about 150 mg-eq. in the deltoid muscle on treatment day 1 and about 100 mg-eq. in the deltoid muscle one week later; and (B) patients switched from the dose of about 6 mg paliperidone ER to paliperidone palmitate using a single day 1 injection of about 150 mg-eq. dose. The results of the simulations were summarized in FIG. 5.

As shown in FIG. 5A, the desired paliperidone plasma levels were maintained during the first week of the switching treatment from about 6 mg paliperidone ER to day 1/day 8 initiation regimen of paliperidone palmitate. Though the palperidone plasma levels decline rapidly from the oral treatment, the plasma levels or concentration increased due to the intramuscular administering of paliperidone palmitate at day 1. Afterward, the administration of the 2^{nd} dose of about 100 mg-eq. dose on day 8 maintained the drug levels in the desired therapeutic range.

On the contrary, the results of FIG. 5B showed that when the day 8 injection was skipped, the paliperidone plasma levels began to decline and became lower than the desired therapeutic range at about 2 weeks after the day1 injection. Therefore, the initiation regimen of day 1/day 8 of paliperidone palmitate provided an effective treatment for switching patients from oral antipsychotics.

In addition to the simulation based analysis, a literature search was performed to evaluate the pharmacokinetic characteristics of other oral antipsychotics. The results of literature search for typical and atypical antipsychotics were summarized in Tables 1 and 2, respectively.

TABLE 1
Terminal Half-life of Oral Typical Antipsychotics

| Oral Typical Antipsychotic | Terminal Half-life | |
|---|---|---|
| Chlorpromazine | 8-35 | hours ^{a} |
| Flupenthixol | 22-36 | hours ^{a} |
| Fluphenazine | 14-24 | hours ^{a} |
| Haloperidol | 12-36 | hours ^{a} |
| Loxapine | 4 | hours ^{b} |
| Molindone | 1.5 | hours ^{b} |
| Perphenazine | 8-21 | hours ^{a} |
| Pimozide | 2-3 | days ^{b} |
| Prochlorperazine | 4-8 | hours ^{b} |
| Thioridazine | 9-30 | hours ^{a} |
| Thiothixene | 34 | hours ^{a} |
| Trifluoperazine | 10-20 | hours ^{b} |

^{a} Ereshefsky L. Pharmacokinetics and drug interactions: update for new antipsychotics. J Clin Psychiatry. 1996; 57 Suppl 11: 12-25.
^{b} “Typical antipsychotic” in Wikipedia: The Free Encyclopedia, Wikimedia Foundation Inc [Encyclopedia on-line]; retrieved Aug. 6, 2009.

TABLE 2
Terminal Half-life of Oral Atypical Antipsychotics

| Oral Atypical Antipsychotic | Terminal Half-life | |
|---|---|---|
| Amisulpride | 12 | hours ^{c} |
| Aripiprazole | 47-68 | hours ^{c} |
| Clozapine | 9-17 | hours ^{c} |
| Olanzapine | 33 | hours ^{c} |
| Paliperidone (9-hydroxy-risperidone) | 25 | hours ^{d} |
| Quetiapine | 6 | hours ^{c} |
| Risperidone active moiety ^{e} | 22 | hours ^{c} |
| Sertindole | 70 | hours ^{c} |
| Ziprasidone | 8-10 | hours ^{c} |

^{c} Mauri M C, Volonteri L S, Colasanti A, Fiorentini A, De Gaspari I F, Bareggi S R. Clinical pharmacokinetics of atypical antipsychotics: a critical review of the relationship between plasma concentrations and clinical response. Clin Pharmacokinet. 2007; 46(5): 359-88.
^{d} Vermeir M, Naessens I, Remmerie B, Mannens G, Hendrickx J, Sterkens P, Talluri K, Boom S, Eerdekens M, van Osselaer N, Cleton A. Absorption, metabolism, and excretion of paliperidone, a new monoaminergic antagonist, in humans. Drug Metab Dispos. 2008 April; 36(4): 769-79.
^{e} Active moiety is the sum of parent drug plus it's active metabolite 9-hydroxy-risperidone

As shown in the tables, all oral antipsychotics have half-life of less than about 3 days. Given the short half-life of the oral antipsychotics, the drug levels of the previous oral antipsychotic would be decline rapidly during the first week of initiation with paliperidone palmitate. Additionally, more than about 75% of the drug from the oral therapy would be washed out from the systemic circulation within the first week. These results further supported the simulations that a second loading dose of paliperidone palmitate after 7 days or on the 8^{th} day after the treatment day 1 would attain the paliperidone concentrations within the desired therapeutic range.

### Example 4

### Switch Treatment from Other Long Acting Injectable Antipsychotic

Pharmacokinetic models or simulations were also developed to examine the drug levels when patients were switched from RISPERDAL® CONSTA® to paliperidone palmitate. The modeling also determined whether the treatment with paliperidone palmitate could be initiated at the next scheduled injection of other injectable antipsychotic such as RISPERDAL® CONSTA®.

The models examined patients who were treated with a bi-weekly administration schedule of RISPERDAL® CONSTA® and switched to paliperidone palmitate for about two weeks after their last RISPERDAL® CONSTA® injection. The simulated concentrations of paliperidone from its palmitate ester were added to the active moiety profile from RISPERDAL® CONSTA® using the superposition principles, as RISPERDAL® CONSTA® has the same active moiety as paliperidone palmitate.

Plasma concentrations were simulated with paliperidone palmitate injection at about two weeks after the last RISPERDAL® CONSTA® injection followed by monthly injections of paliperidone palmitate. The simulation models analyzed two scenarios: (A) a low dose scenario where patients were switched from about 25 mg RISPERDAL® CONSTA® to about 50 mg-eq. paliperidone palmitate followed by monthly injections of about 50 mg-eq. paliperidone palmitate; and (B) a high dose scenario where patients were switched from about 50 mg RISPERDAL® CONSTA® to about 100 mg-eq. paliperidone palmitate followed by monthly injections of about 100 mg eq. paliperidone palmitate. These results were summarized in FIG. 6.

FIG. 6 showed that, for both low and high dose cases, the drug levels were maintained close to the steady-state concentrations right after the switch from RISPERDAL® CONSTA® to paliperidone palmitate. Additionally, after the last injection of RISPERDAL® CONSTA®, the steady state concentrations were maintained for about 4-5 weeks and declined thereafter with a mean plasma half-life of about 4-6 days. Therefore, at the time of switching treatment, only a single injection of paliperidone palmitate was sufficient. This simulation indicated that when switching patients from previous treatment of other long-acting injectable antipsychotics, paliperidone palmitate therapy may be initiated in place of the next scheduled injection and continued at monthly intervals. Also, the simulation indicated that the second dose of initiation dosing regimen and oral supplement were not required when switching from other long acting injectable antipsychotics.

In addition to the simulation based analysis, a literature search was conducted to evaluate the pharmacokinetic characteristics of other long acting injectable antipsychotics. The results were summaried in Table 3.

TABLE 3
Summary of the properties of depot intramuscular antipsychotics

| | | |
|---|---|---|
| | Administration | |
| Drug | interval | t_{1/2} ^{f} |

| | | | | |
|---|---|---|---|---|
| Clopenthixol decanoate ^{a} | 2-4 | weeks | 19 | days |
| Perphenazine enanthate ^{a} | 2 | weeks | 4-6 | days |
| Pipothiazine palmitate ^{a} | 4 | weeks | 15-16 | days |
| Haloperidol decanoate ^{a} | 4 | weeks | 21 | days |
| Fluspirilene ^{a} | 1 | week | 7 | days |
| Zuclopenthixol decanoate ^{a} | 2-4 | weeks | 19 | days |
| Flupenthixol decanoate ^{b} | 2-4 | weeks | 17 | days |
| Fluphenazine decanoate ^{b} | 2-5 | weeks | 14 | days |
| Fluphenazine enanthate ^{c} | 1 | week | 4 | days |
| Risperidone Microspheres ^{d} | 2 | weeks | 4-6 | days |
| Olanzapine pamoate ^{e} | 2-4 | weeks | 30 | days |

^{a} Altamura A C, Sassella F, Santini A, Montresor C, Fumagalli S, Mundo E. Intramuscular preparations of antipsychotics: uses and relevance in clinical practice. Drugs. 2003; 63(5): 493-512.
^{b} Kane J M, Aguglia E, Altamura A C, Ayuso Gutierrez J L, Brunello N, Fleischhacker W W, Gaebel W, Gerlach J, Guelfi J D, Kissling W, Lapierre Y D, Lindström E, Mendlewicz J, Racagni G, Carulla L S, Schooler N R. Guidelines for depot antipsychotic treatment in schizophrenia. European Neuropsychopharmacology Consensus Conference in Siena, Italy. Eur Neuropsychopharmacol. 1998; 8(1): 55-66.
^{c} Levron J C, Ropert R. Clinical pharmacokinetics of haloperidol decanoate. Comparison with other prolonged-action neuroleptics. Encephale. 1987; 13(2): 83-7.
^{d} Gefvert O, Eriksson B, Persson P, Helldin L, Björner A, Mannaert E, Remmerie B, Eerdekens M, Nyberg S. Pharmacokinetics and D2 receptor occupancy of long-acting injectable risperidone (Risperdal Consta) in patients with schizophrenia. Int J Neuropsychopharmacol. 2005; 8(1): 27-36.
^{e} Eli Lilly. Zypadhera. Summary of product characteristics. The Netherlands: Eli Lilly Nederland B. V. 2008. Available Online at: http://www.emea.europa.eu/humandocs/PDFs/EPAR/Zypadhera/H-890-PI-en.pdf. Accessed Sep. 1, 2009.
^{f} t_{1/2} = apparent terminal half-life after multiple dosing

The results in Table 3 showed that, for all depot antipsychotics, the administration interval was in the range of about 1-2 half-life for each product. Based on the simple first-order elimination pharmacokinetic principles, it may take about 4 to 5 half-life for such drugs to be eliminated from the systemic circulation. Therefore, there would be sustained therapeutic levels of the prior drug in the systemic circulation when paliperidone palmitate is administered in place of the next scheduled injection of the previous antipsychotic. Given that significant levels of the previous antipsychotic would be present in the systematic circulation, there would be no need to use the 2^{nd} initiation dose of paliperidone palmitate on day 8.

What is claimed is:

1. A dosing regimen for administering an injectable paliperidone palmitate depot to a patient in need of psychiatric treatment that has been treated with a monthly injectable paliperidone palmitate depot, wherein said patient misses the next scheduled maintenance dose of the monthly injectable paliperidone palmitate depot, comprising:
(1) administering intramuscularly in the deltoid muscle of said patient a first reinitiation loading dose of the monthly injectable paliperidone palmitate depot; and
(2) administering intramuscularly in the deltoid or gluteal muscle of said patient a reinitiation maintenance dose of the monthly injectable paliperidone palmitate depot on about the 23rd day to about the 37th day after administering of said first reinitiation loading dose.

2. The method of claim 1, further comprising administering in the deltoid or gluteal muscle of said patient said reinitiation maintenance dose monthly.

3. The method of claim 1, wherein said patient misses the next scheduled maintenance dose of the monthly injectable paliperidone palmitate depot for more than about 4 weeks and less than about 6 weeks.

4. The method of claim 1, wherein said patient misses the next scheduled maintenance dose of the monthly injectable paliperidone palmitate depot for more than about 6 weeks and less than about 6 months.

5. The method of claim 1, wherein said patient misses the next scheduled maintenance dose of the monthly injectable paliperidone palmitate depot for more than about 6 months.

6. The method of claim 3, wherein said first reinitiation loading dose is the same amount as said scheduled maintenance dose.

7. The method of claim 3, wherein said first reinitiation loading dose is about 39 mg to about 234 mg.

8. The method of claim 3, wherein said reinitiation maintenance loading dose is about 39 to about 234 mg.

9. The method of claim 3, wherein said patient is in need of treatment for psychosis.

10. The method of claim 3, wherein said patient is in need of treatment for schizophrenia.

11. The method of claim 3, wherein said patient is in need of treatment for bipolar disorder.

12. The method of claim 4, further comprising administering intramuscularly in the deltoid or gluteal muscle of said patient a second reinitiation loading dose of the monthly injectable paliperidone palmitate depot on about the 6th day to about the 10th day after administering of said first reinitiation loading dose.

13. The method of claim 12, further comprising administering in the deltoid or gluteal muscle of said patient said reinitiation maintenance dose monthly.

14. The method of claim 12, wherein said first reinitiation loading dose is about 39 mg to about 117 mg.

15. The method of claim 12, wherein said second reinitiation loading dose is about 39 mg to about 117 mg.

16. The method of claim 5, further comprising administering intramuscularly in the deltoid or gluteal muscle of said patient a second reinitiation loading dose of the monthly injectable paliperidone palmitate depot on about the 6th day to about the 10th day after administering of said first reinitiation loading dose.

17. The method of claim 16, further comprising administering in the deltoid or gluteal muscle of said patient said reinitiation maintenance dose monthly.

18. The method of claim 16, wherein said first reinitiation loading dose is about 39 mg to about 117 mg.

19. The method of claim 16, wherein said second reinitiation loading dose is about 39 mg to about 117 mg.

20. A dosing regimen for administering an injectable paliperidone palmitate depot to a patient in need of psychiatric treatment that has been treated with injectable antipsychotic drugs other than paliperidone palmitate, wherein said patient is switched from said injectable antipsychotic drugs to injectable paliperidone palmitate depot, comprising:
(1) administering intramuscularly in the deltoid muscle of said patient a first loading dose of said injectable paliperidone palmitate depot; and
(2) administering intramuscularly in the deltoid or gluteal muscle of said patient a maintenance dose of said injectable paliperidone palmitate depot on about the 23rd day to about the 37th day after administering of said first reinitiation loading dose.

21. The method of claim 20, further comprising administering in the deltoid or gluteal muscle of said patient said maintenance dose monthly.

22. The method of claim 20, wherein said first loading dose is about 78 mg to about 234 mg.

23. The method of claim 20, wherein said maintenance dose is about 39 mg to about 234 mg.

24. The method of claim 20, wherein said patient is in need of treatment for psychosis.

25. The method of claim 20, wherein said patient is in need of treatment for schizophrenia.

26. The method of claim 20, wherein said patient is in need of treatment for bipolar disorder.
